(19) United States
(12) Patent Application Publication
(10) Pub. No.: US 2024/0199628 A1
(43) Pub. Date: Jun. 20, 2024

(54) TETRAPYRROLE-BASED COMPOUNDS AND THEIR FOMULATIONS FOR ANTI-MICROBIAL THERAPY

(71) Applicants: biolitec Unternehmensbeteiligungs II AG, Vienna (AT); Fraunhofer Gesellschaft zur Foerderung der AF EV, Munchem (DE)

(72) Inventors: Volker Albrecht, Nuhetal (DE); Hagen Von Briesen, Hunstetten (DE); Dietrich Scheglmann, Jena (DE); Burkhard Gitter, Jena (DE); Arno Wiehe, Berlin (DE); Sylvia Wagner, Neunkirchen (DE); Anja Germann, Saarbrucken (DE); Dorika STEEN, Jena (DE); Gerhard Wieland, Jena (DE)

(21) Appl. No.: 18/295,582

(22) Filed: Apr. 4, 2023

(30) Foreign Application Priority Data
Oct. 5, 2021 (WO) PCT/EP2021/077470

(51) Int. Cl. C07D 487/22; A61K 31/315; A61K 31/407; A61P 31/04; A61P 31/18; C07F 3/06

(52) U.S. Cl. CPC C07D 487/22; A61K 31/315; A61K 31/407; A61P 31/04; A61P 31/18; C07F 3/06

(57) ABSTRACT

Biologically active compounds are provided that may be used as photosensitizers for diagnostic and therapeutic applications, particularly for phototherapy, including photodynamic therapy, of viral or bacterial infections. As the compounds exhibit also toxicity against targets (bacteria, viruses) without light these biologically active compounds may also be used for the light-independent treatment of such diseases. Embodiments include porphyrins, chlorins, and dihydroxy chlorins as well as their zinc complexes. Also included are embodiments where these compounds are incorporated into liposomal formulations.

### CROSS REFERENCE TO PRIORITY APPLICATION

This patent application claims priority to U.S. provisional patent application No. 63/087,674, filed on Oct. 5, 2020, by Volker Albrecht et al. entitled, “TETRAPYRROLE-BASED COMPOUNDS AND THEIR FORMULATIONS FOR ANTI-MICROBIAL THERAPY”, which is hereby expressly incorporated by reference in its entirety as part of the present disclosure.

### TECHNICAL FIELD

The invention relates to the chemistry of biologically active compounds. More particularly it relates to certain tetrapyrrole-based compounds and their zinc complexes that can be used to treat bacterial and viral infections. The action of these tetrapyrrole-based compounds and their zinc complexes against bacteria and viruses may be intensified by light, thus they can also be used as photosensitizers for a wide range of light irradiation treatments such as photodynamic therapy of infections and other diseases.

### BACKGROUND

Bacterial and viral infections present one of the main challenges in medical practice and are one of the main causes of death worldwide. Though a large number of antibacterial (antibiotic) and antiviral treatments are known and numerous compounds with antibiotic and antiviral action have been identified there is still need for new active substances, formulations and therapies that can be applied to bacterial and viral infections which cannot successfully be treated by known compounds and therapies. This is particularly true in the light of growing antibiotic resistance, which renders many known antibiotics ineffective against bacterial infections [1]. Similar to antibiotic resistance, resistance to antiviral drugs is also a current cause of concern [2]. Thus, there is also need for new therapeutic options that rely on different mechanisms other than conventional antibiotics and antivirals. One of those newer therapeutic approaches is antimicrobial photodynamic therapy (aPDT) which apart from being used against bacteria and other microbiota can also be applied against viruses [3]. Photodynamic therapy (PDT) in general is already being explored for use in a variety of medical applications [4], and particularly is a well-recognized treatment against tumors [5]. Photodynamic therapy uses light and a photosensitizer to achieve its desired medical effect. A large number of naturally occurring and synthetic dyes have been evaluated as potential photosensitizers for aPDT and PDT. Perhaps the most widely studied class of photosensitizers are tetrapyrrolic macrocyclic compounds. Among them, especially porphyrins and chlorins have been tested for their photodynamic efficacy.

The photodynamic effect is only observed if the three necessary components, the photosensitizer, light and oxygen (which is present in the cell) are present at the same time [4]. The selective application of light in PDT restricts aPDT and PDT in general to the local treatment area in contrast to the systemic action of many other drugs and treatments.

aPDT has up to now mainly been applied for localized bacterial infections. Bacteria are generally divided into two main groups based on the different properties and construction of their cell walls, i.e. gram positive and gram negative bacteria. From these, especially gram negative bacteria are most resistant to antibacterial treatments, due to their complex cell wall. For viruses there are a number of different classifications, depending on the type of their genetic information (RNA or DNA viruses) or the division into enveloped and non-enveloped viruses.

### SUMMARY

Embodiments include biologically active compounds that can be used as photosensitizers for a wide range of applications including light irradiation treatments of bacterial and viral infections. One of the limitations of current aPDT and PDT is the localized effect of the treatment, which is due to the fact, that light has to be delivered to the treatment site. This could be overcome by compounds which act as photosensitizers but additionally exhibit a light-independent toxicity against e.g. bacteria or viruses. Therefore, the structures described herein are active as photosensitizers but may also be used for a systemic treatment due to their light-independent toxicity against e.g. bacteria or viruses. In addition, due to their light-absorbing and light-emitting properties these compounds may also be employed for diagnostic purposes e.g. by detecting their luminescence.

Embodiments include chemically stable tetrapyrrole compounds and tetrapyrrole zinc complexes, namely porphyrins, chlorins and dihydroxychlorins useful for various medical applications such as the photodynamic therapy of bacteria and viruses. Yet, these compounds may also be used for the treatment of these diseases without having to administer light, thereby also enabling a systemic treatment.

Embodiments include tetrapyrroles and tetrapyrrole zinc complexes incorporating 3-hydroxyphenyl-substituents that can be used in the photodynamic therapy of viral or bacterial infections. These compounds may also be used in the therapy of viral or bacterial infections without the necessity to administer light. Still, these compounds may also be used in light-based diagnostics of such diseases. If used in combination with light the compounds can benefit from the different absorption properties of porphyrins and chlorins enabling efficient irradiation at longer wavelengths thereby allowing treatment of deeper lesions as e.g. in the case of local bacterial infections.

Embodiments include pharmaceutically acceptable formulations for the biologically active compounds herein described, such as liposomal formulations to be injected avoiding undesirable effects like precipitation at the injection site or delayed pharmacokinetics of the compounds. Embodiments also include liposomal formulations incorporating polyethylene-glycol modified lipids which enable a longer circulation time in the blood thereby enhancing the probability of interaction with the target entities.

Briefly stated, embodiments include biologically active compounds and methods to obtain biologically active compounds that can be used for the treatment of bacterial or viral infections. Additionally, these compounds may also—in combination with light irradiation—be employed as photosensitizers for diagnosis and treatment of viral or bacterial infections.

The above and other objects, features, and advantages of the present invention will become apparent from the following description read in conjunction with the accompanying figures.

### BRIEF DESCRIPTION OF FIGURES

FIG. 1A illustrates the antibacterial effect of [5,10,15,20-tetrakis(3-hydroxyphenyl)porphyrinato]zinc(II) against S. aureus.

FIG. 1B illustrates the antibacterial effect of [5,10,15,20-tetrakis(3-hydroxyphenyl)porphyrinato]zinc(II) against K. pneumoniae.

FIG. 1C illustrates the antibacterial effect of [5,10,15,20-tetrakis(3-hydroxyphenyl)porphyrinato]zinc(II) against E. aerogenes.

FIG. 1D illustrates the antiviral effect of [5,10,15,20-tetrakis(3-hydroxyphenyl)porphyrinato]zinc(II) against HIV-1 infectious molecular env clones.

FIG. 1E illustrates the antibacterial effect of [5,10,15,20-tetrakis(3-hydroxyphenyl)porphyrinato]zinc(II) against A. baumannii.

FIG. 1F illustrates the antibacterial effect of [5,10,15,20-tetrakis(3-hydroxyphenyl)porphyrinato]zinc(II) against biofilms of S. aureus.

FIG. 1G illustrates the antibacterial effect of a liposomal formulation of [5,10,15,20-tetrakis(3-hydroxyphenyl)porphyrinato]zinc(II) against S. aureus.

### DETAILED DESCRIPTION OF EMBODIMENTS

Embodiments include biologically active compounds that may be used as antibacterial or antiviral compounds or—in combination with irradiation by a suitable light source corresponding to the absorption spectrum of the compound—as photosensitizers for the treatment of viral or bacterial infectious diseases. Due to their light-independent toxicity they may also be used for the therapy of such diseases without the necessity to administer light. The compounds described herein have the advantage that they are easily produced and characterized. Embodiments also include methods to formulate compounds of the present disclosure to ease their administration and increase their selectivity and thus therapeutic efficacy. The compounds herein described enhance the effectiveness of biologically active compounds compared to the compounds described in prior art, by allowing to combine aPDT and conventional antiviral or antibacterial treatment and by custom-made pharmacokinetic behavior, depending on the particular application. Moreover, the compounds according to present disclosure may be loaded onto the surfaces of medical devices to provide an antiviral or antibacterial effect, or to aid in visualization in diagnostic tools. The compounds may be loaded with conventional techniques known in the art.

The compounds of the present disclosure can benefit from a specific interaction with components of viruses specifically the viral envelope, e.g. with proteins on the viral surface or viral RNA/DNA. Thus, they can exert their antiviral activity by interfering with one or more stages of the viral replication cycle. However, apart from this light-independent activity the compounds described herein can also serve as photosensitizers which allows them to benefit from the specific features of PDT and aPDT. Upon irradiation the photosensitizer undergoes a well-known photochemistry which results in the formation of reactive oxygen species (ROS, e.g. singlet oxygen, hydroxyl radicals, hydrogen peroxide, superoxide anion radical). These ROS inactivate viruses and bacteria by oxidatively damaging bacterial and viral components, like DNA or RNA (for viruses), lipids or proteins. Thus, the action of the ROS is relatively unspecific. However, this ‘unspecificity’ of the action of ROS generated by the photosensitizer is seen as an advantage, because it makes resistance development against an aPDT treatment unlikely [3]. Given the genetic flexibility of viruses and bacteria and the big challenge of resistance formation against conventional antibiotics, the compounds of the present disclosure have the advantage of a dual action mechanism.

The synthesis of the compounds of the present disclosure used for inactivating viruses and bacteria, relies on methods known in the art. They can be synthesized by first reacting pyrrole and an aldehyde with a protected hydroxy group (e.g. 3-methoxy benzaldehyde or 3-acetoxybenzaldehyde) in a condensation reaction to form the substituted tetrapyrrole (porphyrin) [6]. At this stage the protective groups are removed (by treatment with e.g. borontribromide to remove the methyl group or a base to remove the acetyl groups) [7]. In the next step the porphyrin is either reduced to the chlorin by in situ generated diamine [7] or oxidatively dihydroxylated by treatment with osmium tetraoxide [8]. Finally, insertion of zinc is accomplished by treating the porphyrin, chlorin or dihydroxy chlorin with a suitable zinc salt, e.g. zinc acetate [9].

In a preferred embodiment of the present disclosure the compound 5,10,15,20-tetrakis(3-hydroxyphenyl)chlorin (mTHPC, Temoporfin) is used in the treatment of enveloped viruses.

In another preferred embodiment of the present disclosure the tetrapyrrole derivatives and their zinc complexes are formulated as a liposomal formulation.

In a specifically preferred embodiment of the present disclosure the compound is 5,10,15,20-tetrakis(3-hydroxyphenyl)chlorin (mTHPC, Temoporfin) used in the treatment of AIDS, Dengue fever or Covid-19.

In yet another preferred embodiment of the present disclosure the compound is [5,10,15,20-tetrakis(3-hydroxyphenyl)porphyrinato]zinc(II) used in the treatment of localized bacterial infections.

Acceptable starting materials for the synthesis of the tetrapyrrole derivatives or their zinc complexes described herein may be pyrrole and an aldehyde with a protected hydroxy group. Suitable methods for the condensation of pyrrole and a substituted benzaldehyde to a porphyrin have long been known in the art [6]. The obtained porphyrin may then be converted to the corresponding chlorin by reduction [7] or to the corresponding dihydroxy chlorin by oxidation by methods known in the art [8]. At this stage or earlier the protective groups can be removed to obtain the free hydroxy groups [7]. At the final stage of the porphyrin, chlorin or dihydroxy-chlorin the tetrapyrrole derivatives can be converted to their zinc complexes by reaction with a suitable zinc salt, e.g. zinc acetate [9].

Example 1.1 shows the preparation of a liposomal formulation of the photosensitizer 5,10,15,20-tetrakis(3-hydroxyphenyl)chlorin (mTHPC, compound 2 of claim 1).

Example 1.2 shows the preparation of a liposomal formulation of the photosensitizer [5,10,15;20-tetrakis(3-hydroxyphenyl)porphyrinato]zinc(II) (mTHPP-Zn, compound 4 of claim 2)

Example 2 shows the preparation of a liposomal formulation of the photosensitizer 5,10,15,20-tetrakis(3-hydroxyphenyl)chlorin (mTHPC, compound 2 of claim 1) using pegylated lipids.

Example 3 shows results of the antibacterial testing of compounds of the present disclosure.

Example 4 shows results of the antiviral testing of the compound 5,10,15,20-tetrakis(3-hydroxyphenyl)chlorin (mTHPC, Temoporfin) of the present disclosure.

Embodiments include tetrapyrrole compounds, free-bases and zinc complexes based on the formulas 1, 2, 3, 4, 5, and 6:

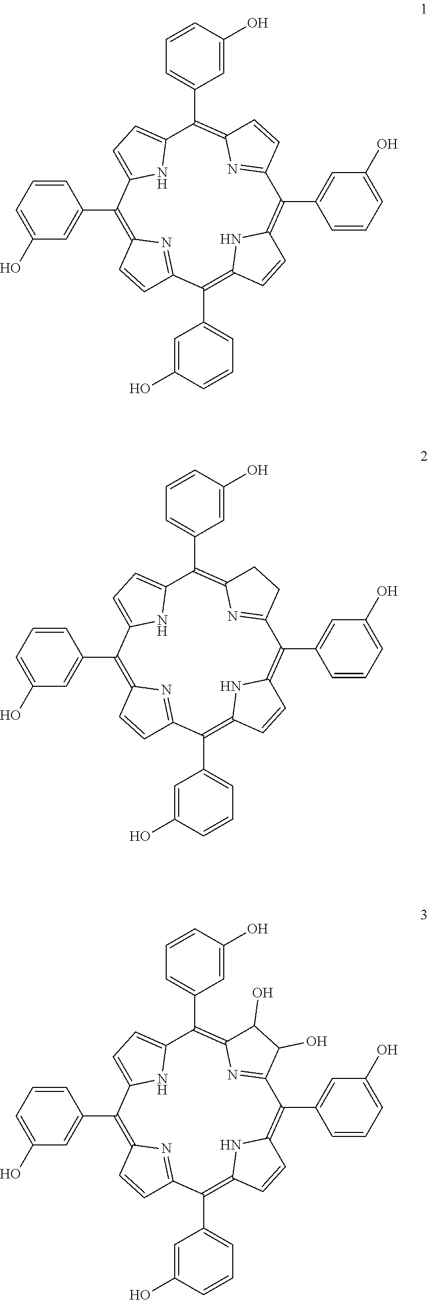 [SMILES: Oc1cccc(-c2c3nc(c(-c4cccc(O)c4)c4ccc([nH]4)c(-c4cccc(O)c4)c4nc(c(-c5cccc(O)c5)c5ccc2[nH]5)C=C4)C=C3)c1 ; Oc1cccc(-c2c3nc(c(-c4cccc(O)c4)c4ccc([nH]4)c(-c4cccc(O)c4)c4nc(c(-c5cccc(O)c5)c5ccc2[nH]5)CC4)C=C3)c1 ; Oc1cccc(-c2c3nc(c(-c4cccc(O)c4)c4ccc([nH]4)c(-c4cccc(O)c4)c4nc(c(-c5cccc(O)c5)c5ccc2[nH]5)C(O)C4O)C=C3)c1]

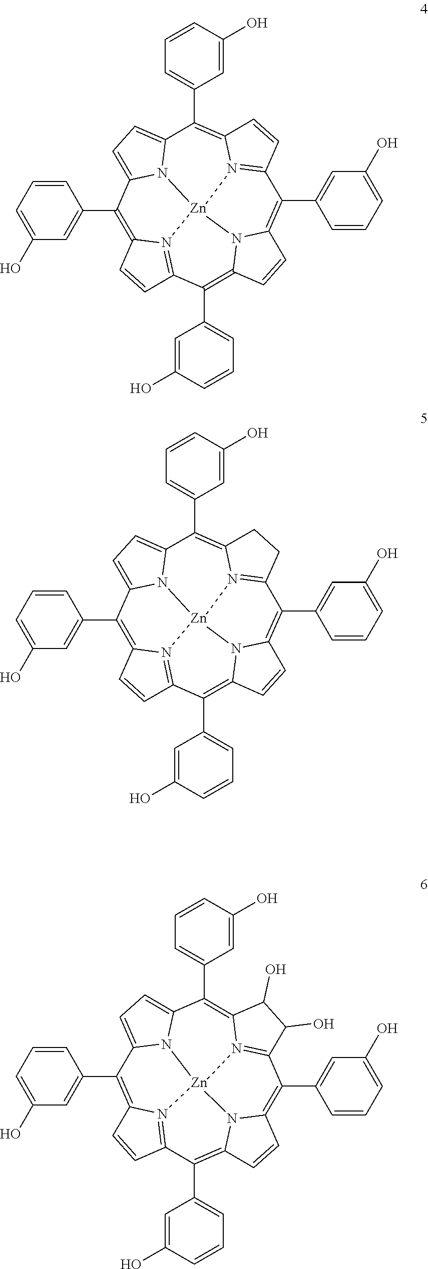

The tetrapyrrole derivatives and their zinc complexes or pharmaceutically acceptable derivatives thereof may be used in therapy and/or in the photodynamic therapy of viral or bacterial infections. The compounds of the present disclosure or pharmaceutically acceptable derivatives thereof may be used in the preparation of a pharmaceutical composition for use in therapy or phototherapy, including photodynamic therapy, of viral or bacterial diseases and infections. In some instances, the compounds of the present disclosure or pharmaceutically acceptable derivatives thereof may be loaded onto a surface of a medical device.

The specifically substituted tetrapyrrole derivatives and their zinc complexes as herein described are suitable to be used for the chemotherapy or phototherapy, including photodynamic therapy, of bacterial and viral infections and diseases. In preferred embodiments the specifically substituted tetrapyrrole derivatives and their zinc complexes as herein described are used in suitable pharmaceutical formulations (e.g. ethanolic solution, ethanol-propylene glycol mixture, liposomal formulations) for the treatment of chronically infected wounds or for the treatment of viral infections with enveloped viruses, like retroviridae (e.g. HIV), coronaviridae (e.g. SARS-CoV-1, SARS-COV-2, MERS-COV) or flaviviridae (e.g. Dengue virus).

In some embodiments, treatment is accomplished by first incorporating the tetrapyrrole derivatives or their zinc complexes into a pharmaceutically acceptable application vehicle (e.g. ethanolic solution, ethanol-propylene glycol mixture, liposomal formulation, or another pharmaceutical formulation) for delivery of the derivatives to the body or to a specific treatment site. Embodiments hence encompass parenteral formulations (like e.g. ethanolic solutions or liposomal preparations) as well as formulations to be administered by direct application to a treatment site (e.g. wounds) or via non-parenteral routes like oral application or local injection. Moreover, embodiments also include formulations that may be applied to the treatment site by specific means e.g. to the lungs via an inhalation device. In case of an aPDT treatment, the treatment area is irradiated with light of a proper wavelength and sufficient power to activate the tetrapyrroles and their zinc complexes for inactivating bacteria or viruses. Due to their amphiphilic nature, the tetrapyrroles and their zinc complexes according to embodiments of the present disclosure may be prepared in various pharmaceutically acceptable and active preparations for different administration routes. In one embodiment such amphiphilic compounds are formulated into liposomes. This liposomal formulation can then be injected avoiding undesirable effects such as precipitation at the injection site or delayed pharmacokinetics. The preparation of such liposomal formulations is exemplified with examples 1 and 2 of the present disclosure. In addition, example 2 uses pegylated lipids for the preparation of the liposomes. Such use of pegylated liposomes enables a longer circulation time of the liposomal carrier in the blood thereby enhancing the probability of interaction with the target entities.

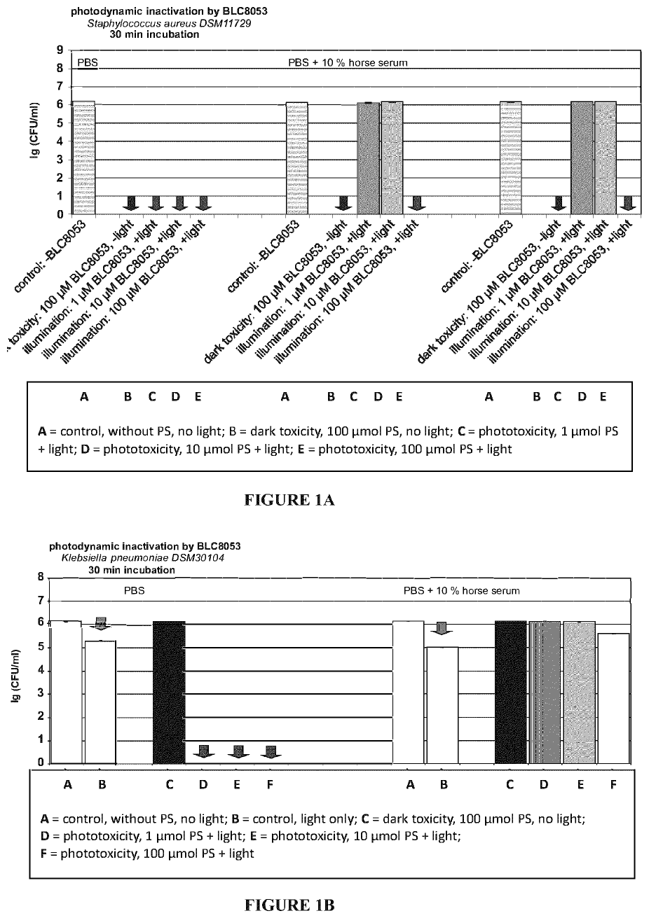

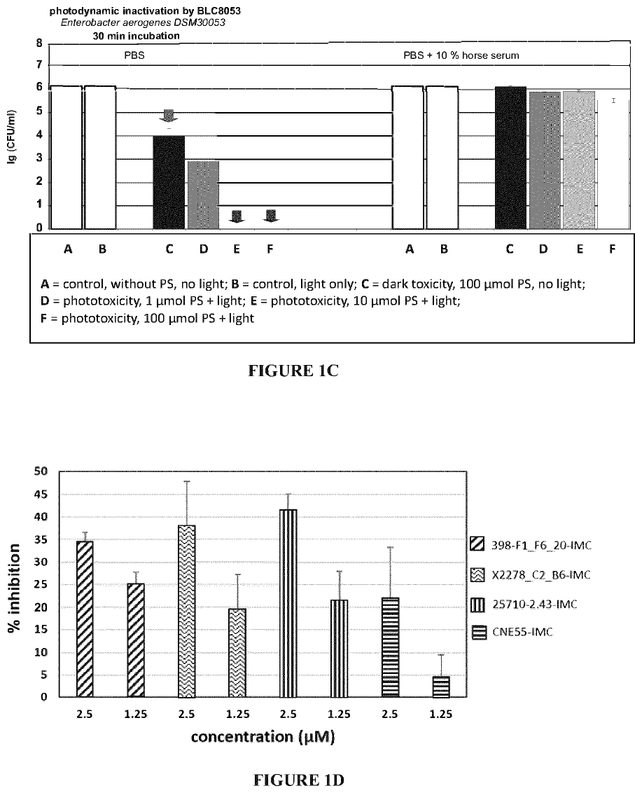

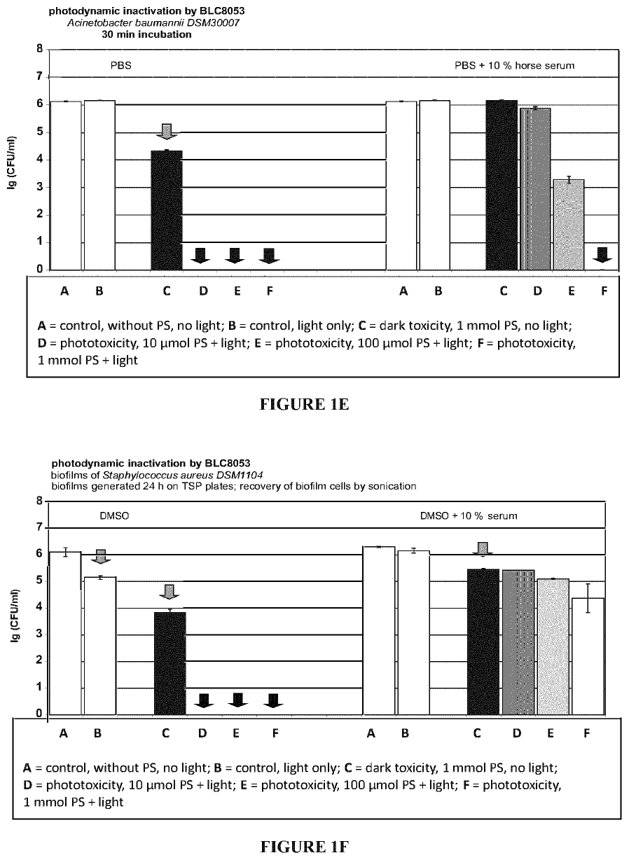

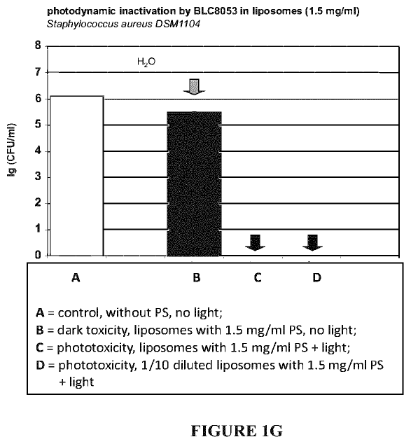

The FIGS. 1A to 1C and 1E to 1G corresponding to example 3.1 exemplarily illustrate the effect of [5,10,15,20-tetrakis(3-hydroxyphenyl)porphyrinato]zinc(II)—compound 4 of claim 2—against bacteria, the gram positive germ Staphylococcus aureus as well as the gram negative germs Klebsiella pneumoniae, Enterobacter aerogenes, and Acinetobacter baumannii proving that these compounds may be used to treat bacterial infections. As can be seen from the examples, [5,10,15,20-tetrakis(3-hydroxyphenyl)porphyrinato]zinc(II) shows a high antibacterial activity against S. aureus even in the absence of light (FIG. 1A) exemplifying the principal suitability of 20) embodiments of the present disclosure for a systemic treatment. This is further supported by the activity of [5,10,15,20-tetrakis(3-hydroxyphenyl)porphyrinato]zinc(II) in the presence of complex media (serum) or even whole blood (FIG. 1A, middle and right). Furthermore, [5,10,15,20-tetrakis(3-hydroxyphenyl)porphyrinato]zinc(II) is also active against Staphylococcus aureus biofilms (FIG. 1F) and when used in a liposomal formulation (FIG. 1G). The antibacterial activity of [5, 10, 15,20-tetrakis(3-hydroxyphenyl)porphyrinato]zinc(II) against gram negative germs is illustrated in FIGS. 1B, 1C, and 1E.

The following examples are presented to provide those of ordinary skill in the art with a full and illustrative disclosure and description of how to make the compounds and formulations of the invention and show their chemotherapeutic and photodynamic activity and are not intended to limit the scope of what the inventor regards as the invention. Efforts have been made to ensure accuracy with respect to numbers used, but some experimental errors and deviations should be accounted for. Also, best measures have been taken to name the compounds with their systematic IUPAC name, nevertheless the basic reference are the given structural formulas.

### Example 1.1

Preparation of a Liposomal Formulation of the Photosensitizer 5,10,15,20-Tetrakis(3-Hydroxyphenyl)Chlorin (mTHPC)

For the preparation of a liposomal formulation, in the first step, all compounds of hydrophobic nature, i.e. lipids and mTHPC are dissolved in organic solvent to obtain a homogenous mixture of all components. After incubation above the transition temperature of the lipids included, the solvent is evaporated with a rotary evaporator, resulting in a thin film on the inner surface of the used glass vessel. This film is then further dried to get rid of all traces of the organic solvent. Once dried, the film is hydrated by adding a watery solution and is incubated again at the same temperature as used in the first step. The obtained mixture is then extruded through polycarbonate membranes of different pore size to obtain a liposomal formulation with liposomes of the average size of 80 to 240 nm in diameter. The formulation may be sterile filtered and preserved by freeze drying.

### Example 1.2

### Preparation of a Liposomal Formulation of the Photosensitizer [5,10,15,20-Tetrakis(3-Hydroxyphenyl)Porphyrinato]Zinc(II)

For the preparation of a liposomal formulation, in the first step, all compounds of hydrophobic nature, i.e. lipids and [5,10,15,20-tetrakis(3-hydroxyphenyl)porphyrinato]zinc(II) are dissolved in organic solvent to obtain a homogenous mixture of all components. After incubation above the transition temperature of the lipids included, the solvent is evaporated with a rotary evaporator, resulting in a thin film on the inner surface of the used glass vessel. This film is then further dried to get rid of all traces of the organic solvent. Once dried, the film is hydrated by adding a watery solution and is incubated again at the same temperature as used in the first step. The obtained mixture is then extruded through polycarbonate membranes of different pore size to obtain a liposomal formulation with liposomes of the average size of 80 to 240 nm in diameter. The formulation may be sterile filtered and preserved by freeze drying.

### Example 2

Preparation of a Liposomal Formulation of the Photosensitizer 5,10,15,20-Tetrakis(3-Hydroxyphenyl)Chlorin (mTHPC) Using Pegylated Lipids

For the preparation of a liposomal formulation, in the first step, all compounds of hydrophobic nature, i.e. (pegylated) lipids and mTHPC are dissolved in organic solvent to obtain a homogenous mixture of all components. After incubation above the transition temperature of the lipids included, the solvent is evaporated with a rotary evaporator, resulting in a thin film on the inner surface of the used glass vessel. This film is then further dried to get rid of all traces of the organic solvent. Once dried, the film is hydrated by adding a watery solution and is incubated again at the same temperature as used in the first step. The obtained mixture is then extruded through polycarbonate membranes of different pore size to obtain a liposomal formulation with liposomes of the average size of 80 to 240 nm in diameter. The formulation may be sterile filtered and preserved by freeze drying.

### Example 3

### Antibacterial Testing

The organisms studied were Staphylococcus aureus DSM 1104 and DSM 11729, gram positive, Klebsiella pneumoniae, gram negative, Enterobacter aerogenes, gram negative, and Acinetobacter baumannii, gram negative.

For antibacterial testing in submerged cultures cultured cells are suspended in sterile phosphate-buffered saline (PBS), sterile PBS supplemented with 10% sterile horse serum, or sterile PBS supplemented with 10% sheep blood. The bacterial suspensions are placed into sterile black well plates with clear bottoms. Concentrations of photosensitizer used in the study were as follows: 1 μM, 10 μM, 100 μM, and 1 mM. After an incubation time period of 30 minutes, the samples are exposed to white light, with a power density and irradiation time resulting in an energy fluency of about 100 J/cm². Control samples contained no photosensitizer and are not exposed to light. The control samples for dark toxicity are only exposed to photosensitizer without any illumination. After irradiation, the samples are removed and suspended again in the culture media. The numbers of colony-forming units (CFU/ml) are enumerated after an adequate incubation time period.

For antibacterial testing of bacterial biofilms the 96 pins of a transferable solid phase plates (TSP; Nunc-Immuno™ TSP with MaxiSorp™ surface) were used to generate the biofilms. The TSP plates were transferred in 96-well plates with 200 μl fresh overnight culture of Staphylococcus aureus DSM1104 cells and incubated at 35° C. about 20 hours by gentle shaking. Within this time the bacterial cells established biofilms on the pins. After removing of unattached cells by gentle washing with 250 μl of 0.9% NaCl solution the TSP plates with the biofilms on the pins were incubated with 250 μl of the photosensitizer solution or the solvent of the photosensitizer solution for control samples for 30 minutes in the dark. After incubation and another washing the pins were irradiated by white light, with a power density and irradiation time resulting in an energy fluence of about 100 J/cm². For the quantification of remaining attached cells, the pins were transferred into 250 μl of 0.9% NaCl solution by gentle sonication. The samples are removed, suspended in the culture media, and the numbers of colony-forming units (CFU/ml) are enumerated after an adequate incubation time period.

The FIGS. 1A to 1C and 1E to 1G corresponding to example 3.1 exemplarily illustrate the effect of [5,10,15,20-tetrakis(3-hydroxyphenyl)porphyrinato]zinc(II)—compound 4 of claim 2—according to embodiments of the present disclosure against bacteria, the gram positive germ Staphylococcus aureus as well as the gram negative germs Klebsiella pneumoniae, Enterobacter aerogenes, and Acinetobacter baumannii. As can be seen from the examples, [5,10,15,20-tetrakis(3-hydroxyphenyl)porphyrinato]zinc(II) shows a high antibacterial activity against S. aureus even in the absence of light (FIG. 1A) exemplifying the principal suitability of embodiments of the present disclosure for a systemic treatment. This is further supported by the activity of [5,10,15,20-tetrakis(3-hydroxyphenyl)porphyrinato]zinc(II) even in the presence of complex media (serum) or whole blood (FIG. 1A, middle and right). Furthermore, [5,10,15,20-tetrakis(3-hydroxyphenyl)porphyrinato]zinc(II) is also active against Staphylococcus aureus biofilms (FIG. 1F) and when used in a liposomal formulation (FIG. 1G). The antibacterial activity of [5,10,15,20-tetrakis(3-hydroxyphenyl)porphyrinato]zinc(II) against gram negative germs is illustrated in FIGS. 1B, 1C, and 1E.

3.1 The antibacterial test of [5,10,15,20-tetrakis(3-hydroxyphenyl)porphyrinato]zinc(II) against S. aureus is shown in FIG. 1A, against K. pneumoniae in FIG. 1B, and against E. aerogenes in FIG. 1C.

3.2 Antibacterial tests of [5,10,15,20-tetrakis(3-hydroxyphenyl)porphyrinato]zinc(II) against A. baumannii are shown in FIG. 1E, against biofilm of S. aureus in FIG. 1F, and against S. aureus by liposomes containing [5,10,15,20-tetrakis(3-hydroxyphenyl)porphyrinato]zinc(II) in FIG. 1G.

### Example 4

### Antiviral Testing

Antiviral testing was performed using temoporfin. The organisms studied were replication competent HIV-1 infectious molecular env clones (IMC) in the A3R5 assay. Following IMC were used: (I) 398-71-F6_20-IMC (origin Tansania, Clade A), (II) X2278_C2_B6-IMC (origin Spain, Clade D), (III) 25710_2.43-IMC (origin India, Clade C) and (IV) CNE55-IMC (origin China, Clade CRF01_AR). Virus specific original env sequences were cloned into the HIV-1 NL4-3 backbone (gene bank ID: M19921) sequence containing a Renilla reniformis luciferase reporter gene to measure the infectivity rate.

A3R5 cells are grown in RPMI1640 supplemented with 10% fetal calf serum, 25 mM HEPES, 50 μg/ml gentamycine and 1 mg/ml geneticin. For virus inhibition studies in A3R5 cells a 2-fold serial dilution of 5,10,15,20-tetrakis(3-hydroxyphenyl)chlorin (mTHPC, Temoporfin) with an initial concentration of 5 μM (confirmed absence of cell cytotoxicity in WST-1—and alamarBlue test at the concentration of 2 μM and a very weak cytotoxicity at 5 μM) were performed in duplicate in two independent experiments. Within the assay the A3R5 cells were added (plus 5 μg/ml DEAE-Dextran) to the temoporfin samples in the presence of the respective amount of IMC at 50,000 RLU equivalents. After 96 hrs incubation the read out of the A3R5 assays occurred. The decrease of the luminescence compared to the virus control was measured using Victor X3 luminometer (Perkin Elmer) and allowed the detection of a possible inhibitory effect of temoporfin against HIV-1 IMCs.

As indicated in FIG. 1D for all tested HIV-1 IMCs an anti-HIV activity was observed for 5,10,15,20-tetrakis(3-hydroxyphenyl)chlorin (mTHPC, Temoporfin) in two independent runs at the concentrations of 2.5 μM and 1.25 μM. The highest one occurred against the Clade C virus stock 25710-2.43-IMC with 42% inhibition and the lowest one for the circulation recombinant form CNE55-IMC with 22% inhibition compared to the virus control at the concentration of 2.5 μM. With 35% for the clade A virus 398-F1_F5_20-IMC and 38% for the clade B virus X2278_C2_B6 the anti-HIV-1 effect at 2.5 μM temoporfin was in the same range.

Having described preferred embodiments of the invention with reference to the accompanying examples, it is to be understood that the invention is not limited to the precise embodiments, and that various changes and modifications may be effected therein by skilled in the art without departing from the scope of the invention as defined in the appended claims.

### REFERENCES

[1] D. M. Lin, B. Koskella, H. C. Lin, «Phage therapy: An alternative to antibiotics in the age of multi-drug resistance», World J. Gastrointest. Pharmacol. Ther., 8, pp. 162-173, 2017.
[2] L. Strasfeld, S. Chou, «Antiviral Drug Resistance: Mechanisms and Clinical Implications», Infect. Dis. Clin. North Am., 24, pp. 413-437, 2010.
[3] A. Wiehe, J. M. O'Brien, M. O. Senge, «Trends and targets in antiviral phototherapy», Photochem. Photobiol. Sci., 18, pp. 2565-2612, 2019.
[4] B. W. Henderson, T. J. Dougherty, Photodynamic therapy, basic principles and clinical applications, New York: Marcel Dekker, 1992.
[5] J. G. Moser, Photodynamic tumor therapy. 2nd and 3rd generation photosensitizers, Amsterdam: Harwood Academic Publishers, 1998.
[6] J. S. Lindsey, I. C. Schreiman, H. C. Hsu, P. C. Kearney and A. M. Marguerettaz, «Rothemund and Adler-Longo Reactions Revisited: Synthesis of Tetraphenylporphyrins under Equilibrium Conditions», J. Org. Chem., 52, pp. 827-836, 1987.
[7] R. Bonnett, M. C. Berenbaum, Porphyrins and medicaments for cancer treatment, Patent EP 0337601 B1.
[8] C. Brückner, D. Dolphin, «2,3-vic-Dihydroxy-meso-tetraphenylchlorins from the Osmium Tetroxide Oxidation of meso-Tetraphenylporphyrin», Tetrahedron Lett., 36, pp. 3295-3298, 1995.
[9] D. Aicher, A. Wiehe, C. B. W. Stark, «Synthesis of Glycoporphyrins Using Trichloroacetimidates as Glycosyl Donors», Synlett, 3, pp. 395-398, 2010.

What is claimed is:

1. A tetrapyrrole compound based on the formulas 1, 2 or 3:

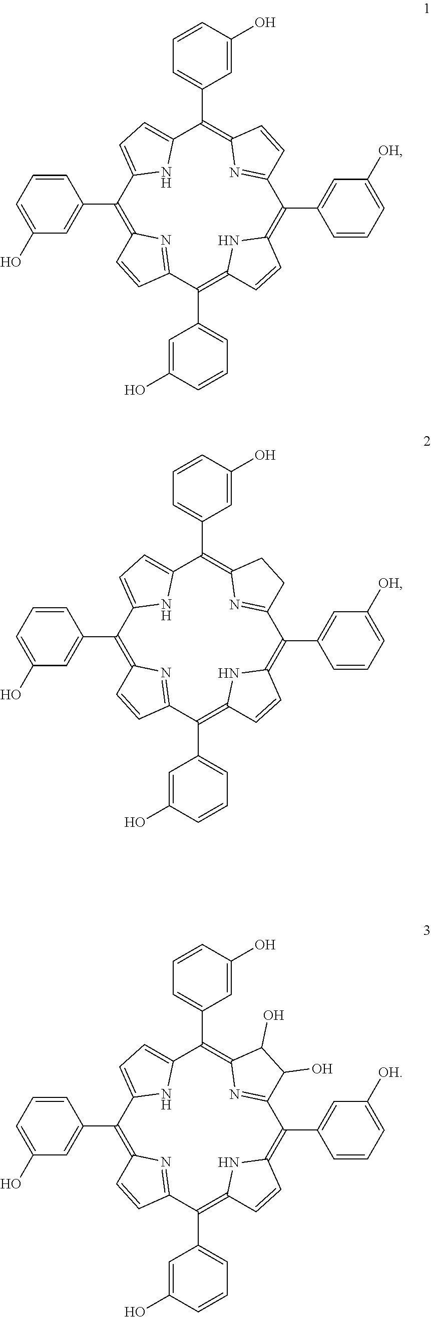 [SMILES: Oc1cccc(-c2c3nc(c(-c4cccc(O)c4)c4ccc([nH]4)c(-c4cccc(O)c4)c4nc(c(-c5cccc(O)c5)c5ccc2[nH]5)C=C4)C=C3)c1 ; Oc1cccc(-c2c3nc(c(-c4cccc(O)c4)c4ccc([nH]4)c(-c4cccc(O)c4)c4nc(c(-c5cccc(O)c5)c5ccc2[nH]5)CC4)C=C3)c1 ; Oc1cccc(-c2c3nc(c(-c4cccc(O)c4)c4ccc([nH]4)c(-c4cccc(O)c4)c4nc(c(-c5cccc(O)c5)c5ccc2[nH]5)C(O)C4O)C=C3)c1]

2. A tetrapyrrole zinc complex based on the formulas 4, 5 or 6:

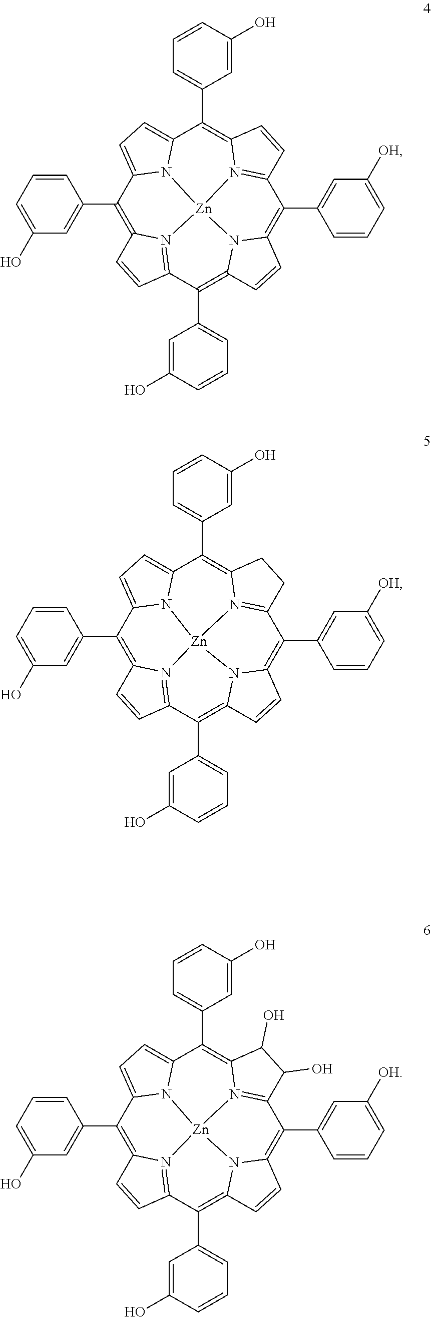

3. The compound of claim 1 or 2 or a pharmaceutically acceptable derivative thereof for use in antimicrobial therapy.

4. The compound of claim 1 or 2 or a pharmaceutically acceptable derivative thereof for use in antibacterial therapy.

5. The compound of claim 1 or 2 or a pharmaceutically acceptable derivative thereof for use in antiviral therapy.

6. The compound of claim 1 or 2 or a pharmaceutically acceptable derivative thereof for use in antiviral therapy against enveloped viruses.

7. The compound of claim 1 or 2 or a pharmaceutically acceptable derivative thereof for use in photodynamic therapy against bacteria or viruses.

8. The compound of claim 1 or 2 or a pharmaceutically acceptable derivative thereof for use in photodynamic therapy against enveloped viruses.

9. The compound of claim 1 or 2 or a pharmaceutically acceptable derivative thereof for use in antiviral therapy against AIDS, Dengue fever or Covid-19.

10. A pharmaceutical formulation of a compound of claim 1 or 2 or a pharmaceutically acceptable derivative thereof for use in antibacterial therapy.

11. A pharmaceutical formulation of a compound of claims 1 and 2 or a pharmaceutically acceptable derivative thereof for use in antiviral therapy.

12. A pharmaceutical formulation of a compound of claims 1 and 2 or a pharmaceutically acceptable derivative thereof for use in antiviral therapy against enveloped viruses.

13. A lipid-based pharmaceutical formulation of a compound of claim 1 or 2 or a pharmaceutically acceptable derivative thereof for use in antiviral therapy against retroviridae (e.g. HIV), coronaviridae (e.g. SARS-COV-1, SARS-COV-2, MERS-COV) or flaviviridae (e.g. Dengue virus).

14. A compound of claim 1 or 2 or a pharmaceutically acceptable derivative thereof when loaded onto a surface of a medical device.
